(19) United States
(12) Patent Application Publication
(10) Pub. No.: US 2019/0350640 A1
(43) Pub. Date: Nov. 21, 2019

(54) MEDICAL SYSTEM, MEDICAL DEVICE AND METHOD FOR OPERATING A MEDICAL SYSTEM

(71) Applicant: OLYMPUS WINTER & IBE GMBH, Hamburg (DE)

(72) Inventor: Michael WOLTER, Hamburg (DE)

(73) Assignee: OLYMPUS WINTER & IBE GMBH, Hamburg (DE)

(21) Appl. No.: 16/482,818

(22) PCT Filed: Mar. 20, 2018
(86) PCT No.: PCT/EP2018/056955

§ 371 Date: Aug. 1, 2019

(30) Foreign Application Priority Data
Mar. 21, 2017 (DE) 10 2017 106 096.0

(51) Int. Cl. A61B 18/14

(52) U.S. Cl. CPC A61B 18/14; A61B 2018/00708

(57) ABSTRACT

A medical system includes an apparatus for generating at least one activation signal, and a medical device, wherein the medical device further includes an apparatus which receives the at least one activation signal and is arranged and configured to activate and/or deactivate the medical device when the apparatus for receiving the at least one activation signal detects a repetition of a predefined activation signal and/or activation signal sequence and/or the repetition thereof and/or a predefined activation signal duration within a predefined time period.

The invention relates to a medical system, a medical device and a method for operating a medical system.

Medical systems, medical devices and methods for operating medical systems can, for example, be used in surgical procedures. In surgical procedures, especially when electrosurgical devices are used, tissue can be gently separated, scabbed and hemorrhaging can be stopped through the effect of heat and/or by means of ultrasound. These procedures include for example procedures for tumor removal, laparoscopic and other endoscopic procedures and procedures using a laser, for example in ophthalmology, or procedures using an ultrasonic scalpel. During surgical procedures, medical devices must often be activated and/or deactivated, wherein said activation and/or deactivation depends in particular on other elements and/or method steps and/or framework conditions. In order to simplify and/or improve surgical procedures and/or medical systems, further improvements with regard to the activation and/or deactivation are desirable.

It is therefore an object of the present invention to provide an improved medical system, an improved medical device and an improved method for operating a medical system. It is in particular the object of the present invention to provide a medical system, a medical device and a method for operating a medical system which may be used flexibly and/or are simplified and/or allow an improved and/or more reliable operation of medical systems and/or devices.

Pursuant to the invention, this object is achieved by means of a medical system comprising an apparatus for generating at least one activation signal, and a medical device, wherein the medical device further comprises an apparatus which receives the at least one activation signal and is arranged and configured to activate and/or deactivate the medical device when the apparatus for receiving the at least one activation signal detects a repetition of a predefined activation signal and/or activation signal sequence and/or repetition thereof and/or a predefined activation signal duration within a predefined time period.

The apparatus for generating at least one activation signal may for example be a generator, in particular a high-frequency (HF) generator and/or an ultrasonic generator.

In this context, activation signal means a signal that can be used for activating and/or deactivating a medical device. In some parts of the present description which focus on the deactivation of a medical device, the activation signal may also be referred to as a deactivation signal. The same applies accordingly to the activation signal sequence and/or activation signal duration.

Activation and/or deactivation may, in particular, also mean controlling the medical device. Preferably, the medical device is configured such that it can be activated and/or deactivated in dependence on the activation signal, and is preferably controllable on the basis of the activation signal.

Preferably, the medical system makes it possible to activate and/or deactivate the medical device automatically in dependence on the activation signal. Especially the provision of the apparatus for receiving the at least one activation signal makes it possible to automatically activate the medical device, namely in dependence on an activation signal. This has the advantage that additional work or handling steps required for a manual activation may be omitted.

The activation signal and/or the activation signal sequence and/or the activation signal duration may preferably be predefined. This means in particular that the apparatus for receiving the at least one activation signal will be able to detect it if (preferably, only if) a received signal corresponds to a repetition of a predefined activation signal and/or to a predefined activation signal sequence and/or the repetition thereof and/or to a predefined activation signal duration. Thus, it can be ensured that an activation and/or deactivation will only take place in dependence on a desired, namely predefined, activation signal. Preferably, the predefinition can be set and/or changed and/or predefined by a user.

A predefined duration is preferably a time period in which a repetition of the activation signal and/or the occurrence and/or the repetition of the activation signal sequence can be expected. Preferably, the predefined duration may be set and/or changed and/or predefined by a user. An activation signal duration is, by way of trial, a time period, in which an activation signal is generated, in particular without interruption.

An activation signal may, for example, be a signal that a generator outputs during operation, in particular in a specific operating mode. An activation signal may, for example, be an acoustic signal and/or an optical signal and/or a radio signal.

Preferably, the medical device is an apparatus for removing surgical smoke by way of suction, which is configured to remove smoke gas by way of suction when it is in an activated state. The medical device may also be an illumination device which is configured to provide, when it is in an activated state, a special light that improves vision. Furthermore, the medical device may have a blinking light that is configured to indicate to a user that the medical device has been activated and/or that another element of the medical system, for example a generator, will be activated if the switch is continued to be actuated. The blinking light may for example fulfill a warning function. Furthermore, the medical device may be a controller for a charging device or have such a controller that is configured to charge, when it is in an activated state, an energy source, for example an energy-based surgical instrument. Furthermore, the medical device may be a video recording apparatus for recording video or photo data or have a video recording apparatus. Furthermore, the medical device may have an audio recording apparatus for recording audio data, in particular voice data, or have an audio recording apparatus. Moreover, the medical device may be a temperature regulating apparatus that is configured to heat and/or cool an energy-based surgical instrument and/or an endoscope. The medical device may also have such a temperature regulating apparatus. Furthermore, the medical device may be a control unit that is configured to control other external apparatuses, such as a computer, a mobile telephone, a tablet computer or the like, when it is in an activated state. The control unit may, for example, also be configured to control a pump, for example for coolant supply.

Preferably, the apparatus for receiving the at least one activation signal is arranged and configured to activate and/or deactivate the medical device when the apparatus for receiving the at least one activation signal does not detect a repetition of an activation signal and/or an activation signal sequence and/or a repetition thereof and/or a predefined activation signal duration within a predefined time period.

This embodiment is in particular preferred when an activation signal and/or an activation signal sequence and/or a predefined activation signal duration are only output during and/or at the beginning of the operation of an electrosurgical instrument and/or during/or at the beginning of the performance of a surgical procedure.

Repetition within the scope of this application means in particular two or more repetitions within the meaning of two or more occurrences of an activation signal and/or an activation signal sequence and/or an activation signal sequence, in particular within a predefined time period.

The repetition of an activation signal and/or of an activation signal sequence may preferably be a regular repetition.

The activation signal may be a periodic signal, wherein it is also possible for several periodic signals to overlap.

The activation signal sequence may comprise signals of different frequencies. Furthermore, a time interval between the signals of the activation signal sequence may be consistent or vary.

The apparatus for receiving the at least one activation signal may, in particular, also be arranged and configured to identify specific and/or different, for example, predefined, signals as an activation signal, and/or to identify specific and/or different, for example predefined, signal sequences as an activation signal sequence.

The apparatus for receiving the at least one activation signal is preferably connected to one or several of the other components of the medical device. The connection may, for example, be realized as a cabled connection via a cable, or as a cable-free or wireless connection. Furthermore, the apparatus for receiving the at least one activation signal is preferably arranged at a distance or able to be arranged at a distance from one or several of the other components of the medical device.

Pursuant to another aspect, the object mentioned above is achieved by means of an apparatus for removing surgical smoke by way of suction, comprising a suction area and a vacuum pump that is arranged and configured to generate a vacuum at the suction area, wherein furthermore the apparatus comprises an acoustic sensor that is arranged and configured to activate the vacuum pump when the acoustic sensor detects a repetition of an acoustic activation signal and/or an acoustic activation signal sequence and/or the repetition thereof within a predefined time period.

Surgical procedures often produce smoke that constitutes a mixture of gaseous, vaporous and particulate substances. In order to prevent or minimize the exposure of medical personnel and patients to this smoke and in order to prevent or reduce an obstruction of vision due to the smoke, automatically or manually activatable apparatuses are used for removing surgical smoke by way of suction. A reliable removal of smoke by way of suction has, especially in case of difficult laparoscopic procedures, the advantage that clear and unobstructed view of the area that is being operated on is ensured. Since the suction of smoke often entails high noise levels, it is preferred to deactivate the smoke suction when it is not needed, and to only activate it when surgical smoke occurs or may occur. Compared to a manual activation (e.g. via a foot switch), an automatic activation of the smoke suction has the advantage that the additional work step required for the activation can be omitted. However, for the automatic activation as well, further improvements are desirable which are provided by the presently described preferred apparatus for removing surgical smoke by way of suction. The presently described preferred apparatus for removing surgical smoke by way of suction is, in particular, able to be used with a plurality of (electro-)surgical apparatuses and systems and/or able to be used in a flexible manner and/or simplified.

A preferred apparatus for removing surgical smoke by way of suction typically comprises a suction area that is preferably moved into the vicinity of the area that is being operated on, in order to be able to remove the surgical smoke occurring in that area by way of suction. The surgical smoke preferably enters the apparatus for removing surgical smoke by way of suction via the suction area. Thus, the suction area can form an inlet for the smoke into the apparatus for removing smoke by way of suction.

Furthermore, a preferred apparatus for removing surgical smoke by way of suction typically comprises a vacuum pump that, when it is in an activated state, typically provides a vacuum required for removing the smoke by way of suction, in particular at the suction area. In a deactivated state, the vacuum pump preferably does not provide a vacuum or only a very small amount of negative pressure at the suction area.

The vacuum pump may be connected to the suction area for example via a connection line, such as a flexible hose. Furthermore, a preferred apparatus for removing surgical smoke by way of suction may have one or several filters by means of which the smoke removed by way of suction can be filtered. Furthermore, a preferred apparatus for removing surgical smoke by way of suction may also have an outlet through which the smoke can exit, preferably in a filtered state. The outlet and/or filter as well may be preferably connected to the vacuum pump and/or the suction area via one or several connection lines, for example via flexible hoses. The vacuum pump may preferably be arranged downstream of the filter. Furthermore, an apparatus for removing surgical smoke by way of suction may also have a, preferably electric, drive unit for powering the vacuum pump.

Furthermore, a preferred apparatus for removing surgical smoke by way of suction may have an insufflation outlet for introducing gas, such as fresh air, filtered smoke or other gases, that may for example be provided via an insufflation inlet, at and/or into the area that is being operated on and/or into a body lumen for maintaining the (air) pressure desired at that location.

Preferably, the preferred apparatus for removing surgical smoke by way of suction has an acoustic sensor. This acoustic sensor is preferably arranged and configured to activate the vacuum pump. The activation of the vacuum pump by the acoustic sensor may take place directly or immediately, or also indirectly or not immediately, for example when the acoustic sensor generates a control signal that activates the vacuum pump. Such a control signal may preferable be an electrical signal.

The provision of an acoustic sensor makes it possible to activate the apparatus for removing surgical smoke by way of suction automatically, namely in dependence on acoustic signals. This has the advantage that additional work or handling steps required for a manual activation may be omitted.

This embodiment is, inter alia, based on the finding that existing options for automatically activating smoke suction have various disadvantages. The activation of smoke gas suction via a detection of the current from the generator through a cable to the electrosurgical instrument requires accessories that must be connected separately to the generator or to the cable. Furthermore, in case of shielded cables, a detection of the current at the cable is not possible or not possible in a reliable manner. When the power plug of the generator is connected directly at the smoke gas suction it is also possible to measure the power consumption of the generator and, in case of a corresponding calibration, to synchronize the suction with the operation of the generator. However, this requires the respective compatible generators which, on the one hand, require a corresponding connection and, on the other, must operate within a certain performance range, in order to allow the calibration of the smoke suction for the synchronization.

The presently described acoustic sensor is therefore preferably arranged and configured to detect a repetition of an acoustic activation signal within a predefined time period. Alternatively or additionally, the acoustic sensor is arranged and configured to detect an acoustic activation signal sequence and/or the repetition thereof within a predefined time period. Alternatively, or additionally, the acoustic sensor is arranged and configured to detect an acoustic activation signal duration within a predefined time period.

An acoustic activation signal may, for example, be a tone output by a generator during the operation and/or at the beginning of the operation of an electrosurgical instrument. An acoustic activation signal sequence may, for example, be a tone sequence output by a generator during the operation and/or at the beginning of the operation of an electrosurgical instrument. Preferably, acoustic activation signals and/or acoustic activation signal sequences may also be other tones and/or tone sequences that are output when a surgical method that might lead to the occurrence of surgical smoke is started and/or being performed.

An acoustic activation signal sequence may for example be a sequence of two or more acoustic signals which is characteristic for the operation and/or the start of the operation of an electrosurgical instrument via a generator and/or for the performance and/or the beginning of a surgical procedure that might lead to the occurrence of surgical gas.

The presently described preferred apparatus for removing surgical smoke by way of suction has, inter alia, the advantage that potential interfering noises, such as the voices of the surgical personnel or background noises, are not recognized as an acoustic activation signal and/or acoustic activation signal sequence, since they are lacking the periodicity within the meaning of a repetition and/or the characteristic features of an activation signal sequence. Furthermore, a functional connection can be detected in this way, for example when a specific acoustic signal sequence and/or a specific repetition pattern of an acoustic signal is recognized as the beginning of a certain (electro-)surgical procedure and/or as the start of a specific operating mode of a generator.

Pursuant to a preferred embodiment, the apparatus for removing surgical smoke by way of suction is characterized in that the acoustic sensor is arranged and configured to deactivate the vacuum pump when the acoustic sensor does not detect a repetition of an acoustic activation signal and/or an acoustic activation signal sequence and/or the repetition thereof within a predefined time period.

In this embodiment, the acoustic sensor deactivates the vacuum pump when no acoustic signal and/or no acoustic signal sequence is detected by the acoustic sensor. This embodiment is in particular preferred when an acoustic activation signal and/or an acoustic activation signal sequence are only output during and/or at the beginning of the operation of an electrosurgical instrument and/or during/or at the beginning of the performance of a surgical procedure that might lead to the occurrence of surgical smoke. This means that the removal of smoke by way of suction is not necessary when no acoustic activation signal and/or the repetition thereof and/or no acoustic activation signal sequence and/or the repetition thereof can be detected and that, therefore, the apparatus for removing surgical smoke by way of suction can be deactivated.

In another preferred embodiment the acoustic sensor is arranged and configured to deactivate the vacuum pump when the acoustic sensor detects a repetition of an acoustic deactivation signal and/or an acoustic deactivation signal sequence and/or the repetition thereof within a predefined time period.

In this embodiment, the vacuum pump is deactivated when an acoustic deactivation signal and/or an acoustic deactivation signal sequence is detected by the acoustic sensor.

An acoustic deactivation signal and/or an acoustic deactivation signal sequence may for example be a tone and/or a tone sequence output by a generator when no electrosurgical instrument is being operated by means of the generator and/or when the generator is in a “stand-by” mode. It is in particular preferred that the acoustic deactivation signal differs from the acoustic activation signal and/or that the acoustic deactivation signal sequence differs from the acoustic activation signal sequence.

In this case, as well, there is, inter alia, the advantage that potential interfering noises, such as the voices of the surgical personnel or background noises, are not recognized as an acoustic deactivation signal and/or acoustic deactivation signal sequence, since they are lacking the periodicity within the meaning of a repetition and/or the characteristic features of a deactivation signal sequence. Furthermore, a functional connection can be detected in this way also in case of the deactivation, for example when a specific acoustic signal sequence and/or a specific repetition pattern of an acoustic signal is recognized as the end of a certain (electro-)surgical procedure and/or as the end of a specific operating mode of a generator.

Repetition within the scope of this application means in particular two or more repetitions within the meaning of two or more occurrences of an acoustic activation signal and/or an acoustic activation signal sequence, in particular within a predefined period of time.

The repetition of an acoustic activation signal and/or an acoustic deactivation signal and/or an acoustic activation signal sequence and/or an acoustic deactivation signal sequence may preferably be a regular repetition.

The activation signal and/or the deactivation signal may be a periodic, preferably a periodic acoustic, signal, wherein it is also possible for several periodic, preferably several periodic acoustic, signals to overlap.

The activation signal sequence and/or the deactivation signal sequence may comprise several periodic acoustic signals of different frequencies. Furthermore, a time interval between the several periodic acoustic signals of the activation signal sequence and/or the deactivation sequence may be consistent or vary.

The acoustic sensor may, in particular, also be arranged and configured to identify specific and/or different, for example, predefined, acoustic signals as an activation signal, and/or to identify specific and/or different, for example predefined, acoustic signal sequences as an activation signal sequence.

Furthermore, the acoustic sensor is preferably arranged and configured to identify specific and/or different, for example predefined, acoustic signals as deactivation signals that differ from the acoustic signals that are to be identified as activation signals. Furthermore, the acoustic sensor is preferably arranged and configured to identify specific and/or different, for example predefined, acoustic signal sequences as deactivation signal sequences that differ from the acoustic signal sequences that are to be identified as activation signal sequences.

Preferably, the acoustic sensor has one or more frequency filters. This makes it possible to limit the apparatus for removing surgical smoke by way of suction to the detection of acoustic signals in one or several frequency ranges, which may, for example, increase the reliability.

Preferably, the acoustic sensor is connected to one or several of the other components of the apparatus for removing surgical smoke by way of suction. The acoustic sensor may be connected to one or several of the other components of the apparatus for removing surgical smoke by way of suction, for example to the vacuum pump. The connection between the acoustic sensor and one or several of the other components of the apparatus for removing surgical smoke by way of suction, in particular the vacuum pump, may for example be realized as a cabled connection via a cable, or as a cable-free or wireless connection.

Furthermore, the acoustic sensor is preferably arranged at a distance or able to be arranged at a distance from one or several of the other components of the apparatus for removing surgical smoke by way of suction, in particular from the vacuum pump. This makes it possible to place the acoustic sensor independently from the area that is being operated on, where preferably in particular the suction area of the apparatus for removing surgical smoke by way of suction is arranged. Preferably, the acoustic sensor is arranged such that the acoustic signals, that are for example generated by a generator for operating electrosurgical devices, can be reliably received by the acoustic sensor. Preferably, the acoustic sensor, in particular a microphone of the acoustic sensor, may be arranged at a generator of an electrosurgical system, in particular directly at the generator.

Pursuant to another aspect, the above mentioned object is achieved by means of a medical device comprising an apparatus for receiving at least one activation signal, that is arranged and configured to activate and/or deactivate the medical device when the apparatus for receiving the at least one activation signal detects a repetition of a predefined activation signal and/or activation signal sequence and/or repetition thereof and/or a predefined activation signal duration within a predefined period of time.

Pursuant to another aspect, the above mentioned object is achieved by means of a method for operating a medical system comprising the provision of an above described medical system, the generation of a repetition of a predefined activation signal and/or activation signal sequence and/or repetition thereof and/or of a predefined activation signal duration within a predefined time period by the apparatus for generating at least one activation signal, the detection of a repetition of a predefined activation signal and/or activation signal sequence and/or the repetition thereof and/or of a predefined activation signal duration within a predefined time period by the apparatus for receiving the at least one activation signal, the activation and/or deactivation of the medical device.

A preferred embodiment of the method comprises further the detection of the absence of a repetition of a predefined activation signal and/or of the absence of an activation signal sequence and/or the repetition thereof and/or of the absence of a predefined activation signal duration within a predefined time period by the acoustic sensor, the activation and/or deactivation of the medial device.

Pursuant to another aspect, the above mentioned object is achieved by means of a method for removing surgical smoke by way of suction, comprising the provision of a above described apparatus for removing surgical smoke by way of suction, detection of a repetition of an acoustic activation signal and/or an acoustic activation signal sequence and/or the repetition thereof within a predefined time period by the acoustic sensor, and the activation of the vacuum pump.

Furthermore, the method for removing surgical smoke by way of suction preferably comprises the detection of the absence of the repetition of an acoustic activation signal and/or of the absence of an acoustic activation signal sequence and/or the repetition thereof within a predefined time period by the acoustic sensor, and the deactivation of the vacuum pump.

Furthermore, another preferred embodiment of the method for removing surgical smoke by way of suction comprises the detection of the repetition of an acoustic deactivation signal and/or of an acoustic deactivation signal sequence and/or the repetition thereof within a predefined time period by the acoustic sensor, and the deactivation of the vacuum pump.

These further aspects and their preferred embodiments have features and/or method steps that make them particularly suitable for being used with a medical system as described herein and/or an apparatus as described herein, in particular with a medical device as described herein and/or an apparatus for removing surgical smoke by way of suction as described herein, and their respective embodiments.

With regard to the advantages, possible ways of realization and realization details of these further aspects and their embodiments, reference is made to the description of the corresponding system and/or apparatus features, above.

Some preferred embodiments of the invention are, by way of example, described by means of the attached figures. The following is shown in

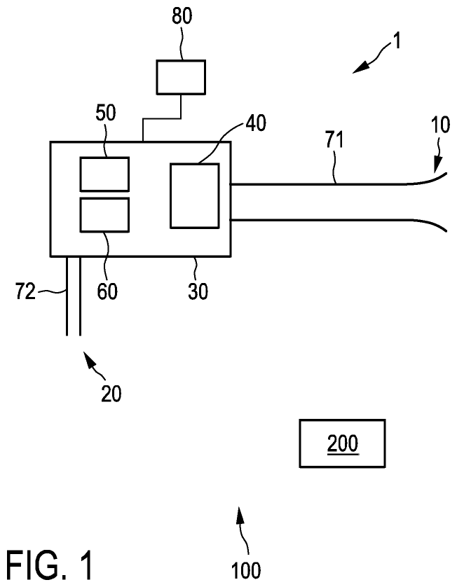

FIG. 1: a schematic illustration of an exemplary embodiment of a medical system;

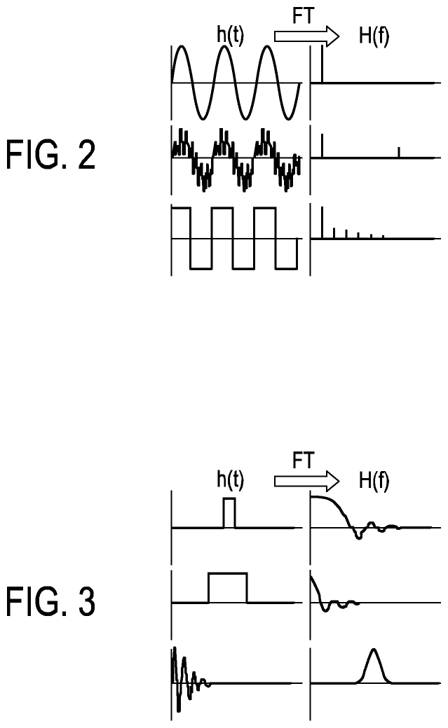

FIG. 2: examples of acoustic activation signals;

FIG. 3: examples of non-periodic acoustic signals;

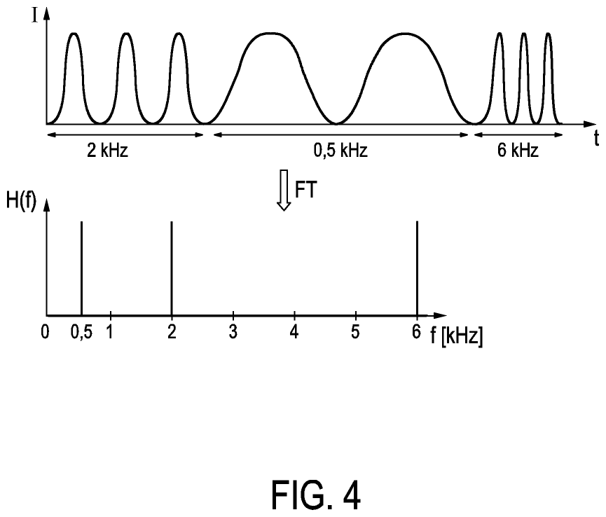

FIG. 4: an example of an acoustic activation signal sequence and the pertaining Fourier transformation;

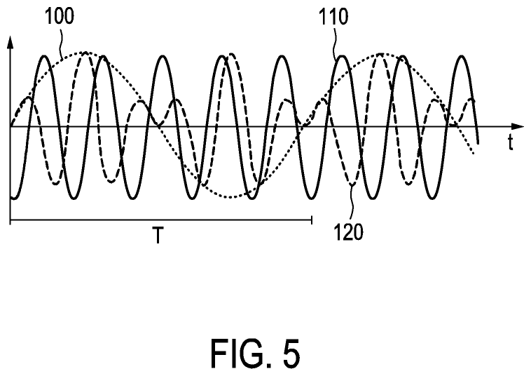

FIG. 5: another example of an acoustic activation signal comprising of several overlapping periodic acoustic signals;

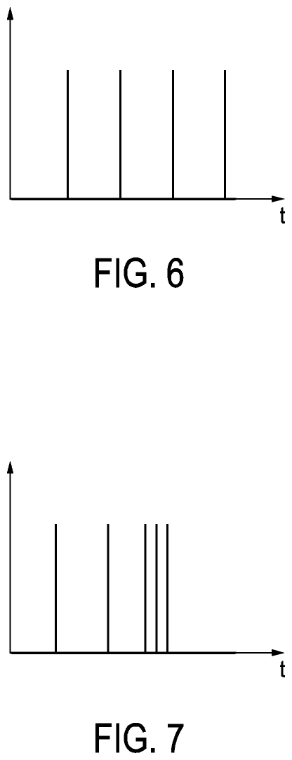

FIG. 6: an example of a regular repetition of an acoustic activation signal;

FIG. 7: an example of an acoustic activation signal sequence with varying time intervals between the individual acoustic signals;

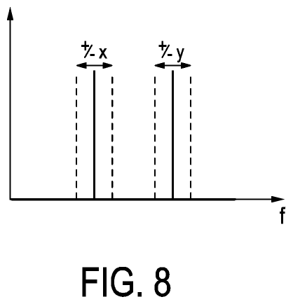

FIG. 8: an example of a predefined frequency tolerance;

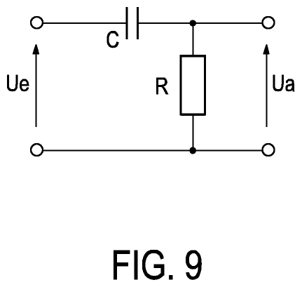

FIG. 9: a schematic illustration of a frequency filter in the form of a high-pass filter;

FIG. 1 shows a schematic illustration of an exemplary embodiment of a medical system 100 comprising an apparatus 200 for generating at least one activation signal, which may, for example, be realized as a high-frequency (HF) generator and/or an ultrasonic generator, and a medical device 1.

In this case, the medical device 1 is realized as an apparatus 1 for removing surgical smoke by way of suction. The apparatus 1 for removing surgical smoke by way of suction has a suction area 10 that a user can move into and/or to an area that is being operated on and/or into a body volume, in order for it to be as close as possible to the place where the surgical smoke is created. The suction area 10 is connected to a vacuum pump 60 powered by an electric drive 50, which may be arranged together with a filter 40 and a functional unit 30, via a connection line 71 in the form of a flexible hose. An outlet 20 for filtered smoke extends away from the functional unit 30 via another connection line 72 in the form of a flexible hose.

In this case, the apparatus 80 for receiving the at least one activation signal is realized as an acoustic sensor 80 and directly or indirectly connected to the other components of the apparatus 1 for removing surgical smoke by way of suction, in particular to the functional unit 30, the vacuum pump 60, the electric drive 50, the filter 40 and the suction area 10, wherein the connection may be realized as a cabled or cable-free connection.

The acoustic sensor 80 is arranged and configured such that it is able to activate and deactivate the vacuum pump 60. This may, for example, be achieved via a, preferably electrical, control signal, that the acoustic sensor 80 transmits to the vacuum pump 60.

Furthermore, the acoustic sensor 80 is arranged and configured to activate the vacuum pump 60 when it detects a repetition of an acoustic activation signal and/or an acoustic activation signal sequence and/or the repetition thereof within a predefined time period. The activation signal and/or the activation signal sequence are preferably generated by the apparatus 200 for the generation of at least one activation signal, for example during the operation of an electrosurgical instrument in a specific operating mode.

Furthermore, the acoustic sensor 80 is preferably arranged and configured to activate the vacuum pump 60 when it does not detect a repetition of an acoustic activation signal and/or an acoustic activation signal sequence and/or the repetition thereof within a predefined time period, i.e. in the absence of the acoustic activation signal and/or of the acoustic activation signal sequence. Alternatively or additionally, the acoustic sensor 80 may be arranged and configured to deactivate the vacuum pump 60 when it detects a repetition of an acoustic deactivation signal and/or an acoustic deactivation signal sequence and/or the repetition thereof within a predefined time period, i.e. when a deactivation signal that differs from the activation signal and/or an acoustic deactivation signal sequence that is different from the acoustic activation signal sequence is detected.

FIGS. 2 and 5 show examples of periodic acoustic activation signals. FIG. 5 shows the fundamental frequency 100 with the period duration T, the 5^{th} harmonic 110 and the product of the fundamental frequency and the 5^{th} harmonic over time t. In principle, periodic acoustic deactivation signals might also look like the signals shown in FIGS. 2 and 5. However, it is particularly preferable that the specific acoustic activation signals and acoustic deactivations signals to be detected differ from each other.

FIG. 3 shows examples of non-periodic acoustic signals, such as noise, background noise, voices, conversations. Such signals are preferably not detected by the acoustic sensor 80; preferably neither as an acoustic activation signal and/or acoustic activation signal sequence, nor as an acoustic deactivation signal and/or acoustic deactivation signal sequence.

FIG. 4 shows an example of an acoustic activation signal sequence and the pertaining Fourier transformation. In this case, an acoustic signal with a frequency of 2 kHz is followed by an acoustic signal with a frequency of 500 Hz, followed by an acoustic signal with a frequency of 6 kHz.

FIG. 6 shows an example of a regular repetition of an acoustic activation signal, and FIG. 7 shows an example of an acoustic signal activation sequence with varying time intervals between the individual acoustic signals. In this case, as well, acoustic deactivation signals and acoustic deactivation signal sequences can in principle look similar to the signals shown in FIGS. 6 and 7, wherein, in this case as well, the specific acoustic activation signals and deactivation signals as well as the specific acoustic activation signal sequences and acoustic deactivation sequences to be detected differ from each other.

FIG. 8 shows an example of a predefined frequency tolerance where a deviation from an acoustic signal in a, preferably predefined, fluctuation range X, Y is also recognized as said acoustic signal. This can increase the recognition accuracy and/or detection reliability.

FIG. 9 shows a schematic illustration of a frequency filter in the form of a high-pass filter, that lets frequencies above a cut-off frequency pass with almost no attenuation and attenuates lower frequencies. FIG. 9 shows the basic structure consisting of a capacitor C and a resistance R, wherein Ue and Ua refer to the input and output voltage. The provision of one or several frequency filters makes it possible to limit the apparatus for removing surgical smoke by way of suction to the detection of acoustic signals in one or several frequency ranges, which may, for example, increase the reliability.

1. A medical system comprising an apparatus for generating at least one activation signal, and a medical device,
wherein the medical device further comprises an apparatus which receives the at least one activation signal and is arranged and configured to activate and/or deactivate the medical device when the apparatus for receiving the at least one activation signal detects a repetition of a predefined activation signal and/or activation signal sequence and/or repetition thereof and/or a predefined activation signal duration within a predefined period of time.

2. A medical system pursuant to claim 1,
wherein the apparatus for receiving the at least one activation signal is arranged and configured to activate and/or deactivate the medical device when the apparatus for receiving the at least one activation signal does not detect a repetition of an activation signal and/or activation signal sequence and/or repetition thereof and/or a predefined activation signal duration within a predefined period of time.

3. A medical system pursuant to claim 1,
wherein the repetition is a regular repetition.

4. A medical system pursuant to claim 1,
wherein the activation signal is a periodic signal, wherein it is also possible for several periodic signals to overlap.

5. A medical system pursuant to claim 1,
wherein the activation signal sequence comprises signals of different frequencies and/or in that a time interval between the signals of the activation signal sequence is constant or varies.

6. A medical system pursuant to claim 1,
wherein the medical device is realized as an apparatus for removing surgical smoke by way of suction.

7. A medical system pursuant to claim 1,
wherein the medical device comprises a suction area and a vacuum pump that is arranged and configured to generate a vacuum at the suction area.

8. A medical system pursuant to claim 1,
wherein the apparatus for receiving the at least one activation signal is realized as an acoustic sensor.

9. A medical system pursuant to claim 1,
wherein the apparatus for receiving the at least one activation signal has a frequency filter.

10. A medical system pursuant to claim 1,
wherein the apparatus for receiving the at least one activation signal is connected to one or several of the other components of the medical device.

11. A medical system pursuant to claim 10,
wherein the connection is realized as a cabled or a cable-free connection.

12. A medical system pursuant to claim 1,
wherein the apparatus for receiving the at least one activation signal is arranged at a distance or able to be arranged at a distance from one or several of the other components of the medical device.

13. A medical device comprising an apparatus for receiving at least one activation signal, that is arranged and configured to activate and/or deactivate the medical device when the apparatus for receiving the at least one activation signal detects a repetition of a predefined activation signal and/or activation signal sequence and/or repetition thereof and/or a predefined activation signal duration within a predefined period of time.

14. A method for operating a medical system, comprising
the provision of a medical system pursuant to claim 1
the generation of a repetition of a predefined activation signal and/or activation signal sequence and/or the repetition thereof and/or of a predefined activation signal duration within a predefined time period by the apparatus for generating at least one activation signal,
the detection of a repetition of a predefined activation signal and/or activation signal sequence and/or the repetition thereof and/or of a predefined activation signal duration within a predefined time period by the apparatus for receiving the least one activation signal,
the activation and/or deactivation of the medical device.

15. A method for operating a medical system, furthermore comprising
the detection of the absence of a repetition of a predefined activation signal and/or the absence of an activation signal sequence and/or the repetition thereof and/or the absence of a predefined activation signal duration within a predefined time period by the apparatus for receiving the least one activation signal,
the activation and/or deactivation of the medical device.
